(19) United States
(12) Patent Application Publication
(10) Pub. No.: US 2019/0261998 A1
(43) Pub. Date: Aug. 29, 2019

(54) ANASTOMOSIS DEVICE

(71) Applicant: Easynotes Ltd., Kfar Truman (IL)

(72) Inventors: Nir Altman, Kfar Etzion (IL); Izhak Fabian, Kfar Truman (IL)

(73) Assignee: Easynotes Ltd., Kfar Truman (IL)

(21) Appl. No.: 15/906,017

(22) Filed: Feb. 27, 2018

(51) Int. Cl. A61B 17/12

(52) U.S. Cl. CPC A61B 17/12009; A61B 2017/00867

(57) ABSTRACT

A system includes a first magnetic anastomosis device including a cover portion in which is disposed a re-shapeable portion. The first magnetic anastomosis device has a contracted orientation when positioned in a delivery device and an expanded orientation after exiting the delivery device. A second magnetic anastomosis device is alignable with the first magnetic anastomosis device for forming a magnetic compression anastomosis.

### FIELD OF THE INVENTION

The present invention generally relates to devices for creating magnet compression anastomosis between organs, such as the gall bladder and portions of the gastrointestinal tract.

### BACKGROUND OF THE INVENTION

Current approaches to surgical treatments for gallbladder disease include open surgical resection, laparoscopic resection, and natural orifice transluminal endoscopic surgery. The risks of these aforementioned techniques include the risks of trans-abdominal and/or transluminal incision (bleeding, infection, perforation, post-operative pain, adhesions, incisional hernia, risks of anesthesia).

Another technique has utilized neodymium magnets for the creation of magnet compression anastomosis between the common bile duct and the small intestine.

U.S. Pat. No. 8,828,032 describes the use of a parent magnet and one or more daughter magnets to create a magnet compression anastomosis. The parent magnet is a permanent rare-earth disc or ring magnet that has an attachment portion (endoscopic clip or suture) to attach the parent magnet to a tissue. The daughter magnets are responsive to the magnetic field of the parent magnet so as to be attracted to the parent magnet through one or more tissues of varying degrees of thickness. The daughter magnets or magnetic materials can include a plurality of ferromagnetic steel ball-bearings or discs, or a magnetic slurry or paste. For example, the daughter magnets can be separate magnetic components that self-assemble into a larger magnetic structure.

### SUMMARY OF THE INVENTION

The present invention seeks to provide an improved device for creating magnet compression anastomosis between organs, such as the gall bladder and portions of the gastrointestinal tract, as is described more in detail hereinbelow. The device includes a combination of a cover portion (e.g., balloon) and a re-shapeable portion (e.g., shape-memory material, such as nitinol). For example, in one embodiment, a balloon for positioning in the body organ (e.g., gall bladder) is filled with magnetic powder or any other kind of magnetic particles or liquid magnetic material (e.g., ferrofluid) and the re-shapeable portion assumes a ring shape after exiting the delivery needle/catheter. Alternatively, the cover portion is made of a flexible magnetic material.

In another aspect, in the delivery needle/catheter, the cover portion has a coiled shape. The re-shapeable portion assumes a geometric shape (e.g., polygonal) after exiting the delivery needle/catheter.

There is thus provided in accordance with an embodiment of the present invention a system including a first magnetic anastomosis device including a cover portion in which is disposed a re-shapeable portion, the first magnetic anastomosis device having a contracted orientation when positioned in a delivery device and an expanded orientation after exiting the delivery device, and a second magnetic anastomosis device alignable with the first magnetic anastomosis device for forming a magnetic compression anastomosis.

### BRIEF DESCRIPTION OF THE DRAWINGS

The present invention will be understood and appreciated more fully from the following detailed description taken in conjunction with the drawings in which:

FIG. 1A is a simplified illustration of an anastomosis device inside a delivery device, in accordance with a non-limiting embodiment of the present invention;

FIG. 1B is a simplified illustration of the anastomosis device starting to exit the delivery device;

FIG. 1C is a simplified illustration of the anastomosis device after exiting the delivery device, in which a re-shapeable portion (e.g., shape-memory device) has assumed a ring shape;

FIG. 1D is a simplified illustration of the anastomosis device aligned with a second magnet for forming a magnetic compression anastomosis;

FIG. 2A is a simplified illustration of an anastomosis device in a coil shape inside a delivery device, in accordance with a non-limiting embodiment of the present invention;

FIG. 2B is a simplified illustration of the anastomosis device exiting the delivery device;

FIG. 2C is a simplified illustration of the anastomosis device after exiting the delivery device, but still in the coil shape;

FIG. 2D is a simplified illustration of the anastomosis device after the re-shapeable portion (e.g., shape-memory device) has assumed a geometric shape;

FIG. 2E is a simplified illustration of the anastomosis device aligned with a second magnet for forming a magnetic compression anastomosis;

FIG. 3A is a simplified illustration of another anastomosis device, in accordance with a non-limiting embodiment of the present invention, in which a series of magnetic elements are wound on or wrapped around a re-shapeable portion and together form a coiled device with one or more coils;

FIG. 3B is a cutaway illustration of the device of FIG. 3A; and

FIG. 3C is another version of the device of FIG. 3A with one coil instead of three coils.

### DETAILED DESCRIPTION OF EMBODIMENTS

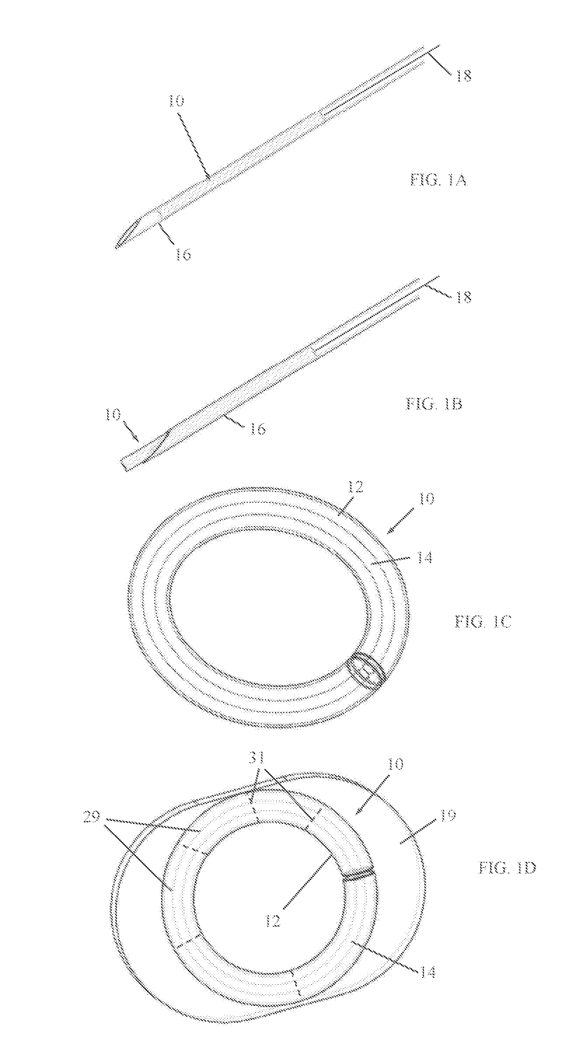

Reference is now made to FIGS. 1A-1D, which illustrate an anastomosis device 10 (also referred to as a first magnetic anastomosis device), in accordance with a non-limiting embodiment of the present invention, constructed and operative in accordance with an embodiment of the present invention.

As seen in FIG. 1C, anastomosis device 10 includes a cover portion 12, such as a balloon, for example, made of a non-magnetic material, such as but not limited to, silicone, latex, rubber or other suitable material, and at least partially filled with magnetic powder (such as but not limited to, samarium cobalt powder, neodymium iron boron powder and the like), magnetic balls or other particles, or liquid magnetic material (e.g., ferrofluid). Alternatively, the cover portion 12 may be made of a flexible magnetic material, such as but not limited to, flexible magnetic sheet or roll (e.g., made of neodymium iron boron). The cover portion 12 may be compressible to fit in a delivery catheter. Alternatively, the cover portion 12 may be inflatable, such as a balloon. Inside the cover portion 12 is disposed a re-shapeable portion 14 (which may be a self-expanding portion), such as but not necessarily, made of a shape-memory material, such as but not limited to, nitinol. The re-shapeable portion 14 may be an elongate member (such as but not limited to, a rod).

In FIG. 1A, the anastomosis device 10 is inside a delivery device 16, such as but not limited to, delivery needle/catheter. In FIG. 1B, the anastomosis device 10 starts to exit the delivery device 16, such as by being pushed distally through the delivery needle/catheter with a detachable push tool 18. The anastomosis device 10 may be delivered percutaneously (or by other suitable means) to the inner portion of an organ, such as into the gall bladder. In FIG. 1C, the re-shapeable portion 14 and the anastomosis device 10 assume a ring shape. In FIG. 1D, the anastomosis device 10 is aligned with a second magnet 19 (second magnetic anastomosis device) delivered and affixed to another body organ (such as a portion of the gastrointestinal tract) for forming a magnetic compression anastomosis between the neighboring organs.

Alternatively, as shown in FIG. 1D in broken lines, the re-shapeable portion 14 may be a plurality of links 29. Each link 29 includes a re-shapeable portion 14 (e.g., rod, bar, wire, etc.) disposed in a cover portion 12. The links 29 can articulate with each other at joints 31, which may be any kind of hinge, coupling, universal joint and the like. This option may be implemented in all embodiments of the invention.

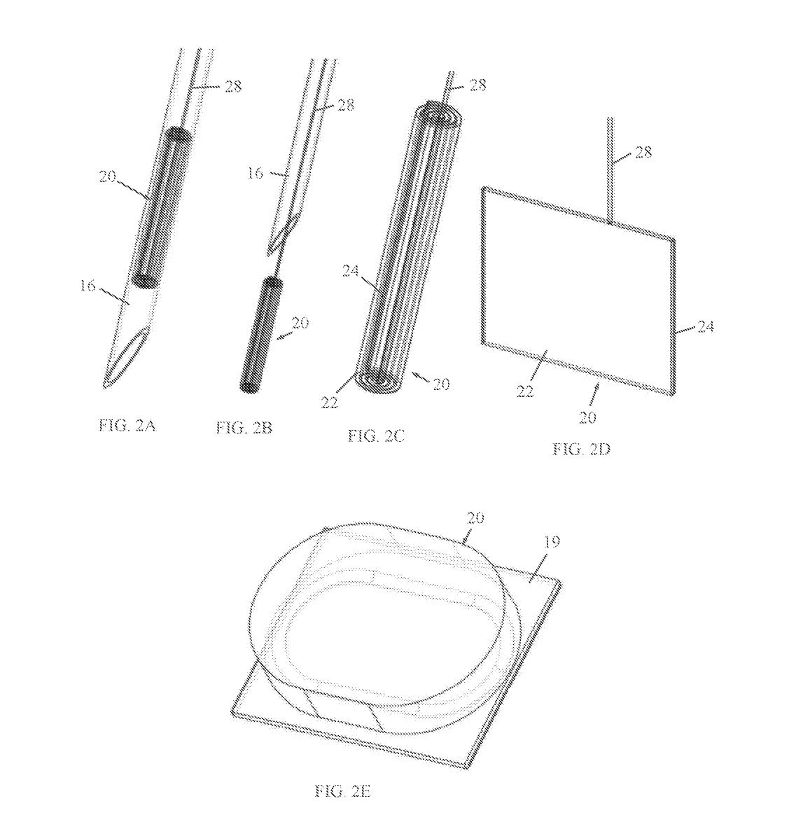

Reference is now made to FIGS. 2A-2E, which illustrate an anastomosis device 20, in accordance with a non-limiting embodiment of the present invention, constructed and operative in accordance with an embodiment of the present invention.

As seen in FIG. 2C, anastomosis device 20 includes a cover portion 22, such as a balloon, as similarly described above (for example, made of a non-magnetic material at least partially filled with magnetic powder, balls, other particles, or liquid magnetic material, or made of a flexible magnetic material). Inside the cover portion 22 is disposed a re-shapeable portion 24 (which may be a self-expanding portion), such as but not necessarily, made of a shape-memory material, such as but not limited to, nitinol.

In FIG. 2A, the anastomosis device 20 is inside the delivery device 16, and has a coil shape. In FIG. 2B, the anastomosis device 20 exits the delivery device 16, such as by being pushed distally through the delivery needle/catheter with a detachable push tool 28. Once again, the anastomosis device 20 may be delivered percutaneously (or by other suitable means) to the inner portion of an organ, such as into the gall bladder. In FIG. 2D, the re-shapeable portion 24 and the anastomosis device 20 assume a geometric shape, such as a four-sided polygon or other polygonal or curved shapes. In FIG. 2E, the anastomosis device 20 is aligned with second magnet 19 delivered and affixed to another body organ (such as a portion of the gastrointestinal tract) for forming a magnetic compression anastomosis between the neighboring organs.

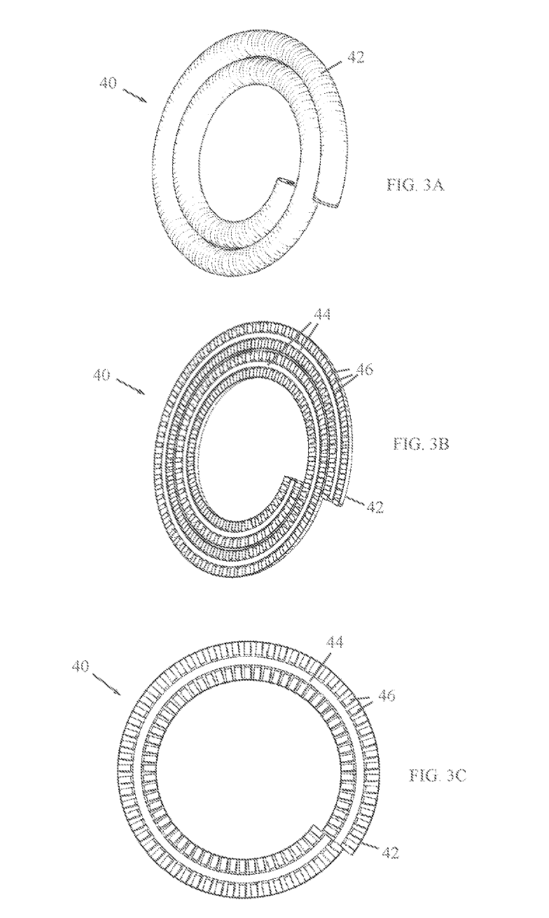

Reference is now made to FIGS. 3A and 3B, which illustrate another anastomosis device 40, in accordance with a non-limiting embodiment of the present invention. Device 40 is another “first magnetic anastomosis device” that can be delivered with delivery device 16 to the body organ and cooperate with second magnet 19 to form an anastomosis, as with the other embodiments.

Device 40 includes a cover portion 42 in which is disposed a re-shapeable portion 44, such as but not limited to a nitinol wire. A series of magnetic elements 46 are wound on or wrapped around re-shapeable portion 44. For example, without limitation, magnetic elements 46 may be beads, chain links or coils and the like, which may be separate, discrete elements or one continuously wound element. The cover portion 42, re-shapeable portion 44 and magnetic elements 46 together form a coiled device 40 with one or more coils. The coils may lie in the same plane or may overlap each other or may assume other shapes. In FIGS. 3A-3B, there are three coils; in FIG. 3C there is one coil. It is noted that in FIG. 3C the ends of the coil are offset from each other; alternatively, they do not have to be offset.

In all embodiments, the re-shapeable portion may be attached to a portion of the cover portion. Alternatively, the re-shapeable portion may not be attached to the cover portion.

In all embodiments, more than one magnetic element may be introduced into the body organ and more than one magnetic element may be delivered to the other side of the body organ.

What is claimed is:

1. A system comprising:
a first magnetic anastomosis device comprising a cover portion in which is disposed a re-shapeable portion, said first magnetic anastomosis device having a contracted orientation when positioned in a delivery device and an expanded orientation after exiting the delivery device; and
a second magnetic anastomosis device alignable with said first magnetic anastomosis device for forming a magnetic compression anastomosis.

2. The system according to claim 1, wherein said cover portion is made of a non-magnetic material at least partially filled with magnetic particles.

3. The system according to claim 1, wherein said cover portion is made of a non-magnetic material at least partially filled with a liquid magnetic material.

4. The system according to claim 1, wherein said cover portion is made of a flexible magnetic material.

5. The system according to claim 1, wherein said re-shapeable portion is attached to a portion of said cover portion.

6. The system according to claim 1, wherein said re-shapeable portion is not attached to said cover portion.

7. The system according to claim 1, wherein said re-shapeable portion assumes a ring shape after exiting the delivery device.

8. The system according to claim 1, wherein said re-shapeable portion assumes a polygonal shape after exiting the delivery device.

9. The system according to claim 1, wherein said cover portion comprises an inflatable portion.

10. The system according to claim 1, wherein said re-shapeable portion comprises a self-expanding portion.

11. The system according to claim 1, wherein said first magnetic anastomosis device comprises magnetic elements wound on or wrapped around said re-shapeable portion.
